(19) United States
(12) Patent Application Publication
(10) Pub. No.: US 2008/0281125 A1
(43) Pub. Date: Nov. 13, 2008

(54) Process for Preparing Enantiopure E-(2S)-Alkyl-5-Halopent-4-Enoic Acids and Esters

(76) Inventors: Markus Rossler, Linz (AT); Gerhard Steinbauer, Enns (AT); Peter Pojarliev, Wien (AT)

Correspondence Address: NIXON & VANDERHYE, PC, 901 NORTH GLEBE ROAD, 11TH FLOOR, ARLINGTON, VA, 22203, US

(21) Appl. No.: 11/885,740

(22) PCT Filed: Feb. 22, 2006
(86) PCT No.: PCT/EP2006/001597

§ 371 Date: May 22, 2008

(30) Foreign Application Priority Data
Mar. 9, 2005 (AT) A 400/2005

(51) Int. Cl. C07C 51/42

(52) U.S. Cl. 562/600

(57) ABSTRACT

A process for preparing enantiopure E-(2S)-alkyl-5-halopent-4-enoic acids and their esters of the formula (I),

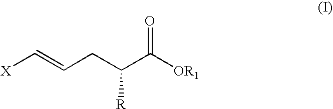

in which R is a C₁-C₆-alkyl radical, R₁ is H or C₁-C₄-alkyl and X is chlorine, bromine or iodine, in which the corresponding racemic 2-alkyl-5-halopent-4-enoic acid
a) is reacted in a suitable solvent first with (S)-3-Methyl-2-phenylbutylamine, quinine or with N-methyl-D-glucamine, after which
b) the corresponding (S)-3-Methyl-2-phenylbutylamine salt, quinine or glucamine salt of the (R)-pentenoic acid is precipitated and removed, and
c) the remaining filtrate is mixed with a second chiral base or an inorganic salt, after which the corresponding salt of the (S)-pentenoic acid is precipitated and
d) is then converted into the corresponding E-(2S)-alkyl-5-halo-4-pentenoic acid and subsequently where appropriate into the corresponding ester of the formula (I) in which R₁ is C₁-C₄-alkyl.

The present invention relates to a process for preparing enantiopure E-(2S)-alkyl-5-halopent-4-enoic acids and their esters in an optical purity of up to e.e. >99% and in a yield of up to 98% of theory.

E-(2S)-Alkyl-5-halopent-4-enoic acids and their esters are valuable intermediates for preparing pharmaceuticals such as, for example, for delta-amino-gamma-hydroxy-omega-arylalkane carboxamides which have renin-inhibiting properties and can be used as antihypertensive agents in pharmaceutical preparations.

One variant for preparing alkyl-5-halopent-4-enoic esters is described for example in WO 01/09079, according to which the desired esters are obtained in a yield of 84% as racemate by reacting isovaleric ester with 1,3-dihalo-1-propene in the presence of a strong base such as, for example, alkali metal amides (LDA). The desired enantiomer is obtained from the racemate by treatment with esterases, for example with pig liver esterase (PLE), in yields of about 32 to 46%.

A substantial disadvantage of this process is the use of the enzyme pig liver esterase (PLE), which is of animal origin.

J. Agric. Food Chem. 32 (1), pp. 85-92, describes for example the preparation of various haloalkene carboxylic acids such as, for example, the racemic 2-isopropyl-5-chloropent-4-enoic acid starting from the corresponding dialkyl isopropylmalonate. The malonate is in this case first alkylated with 1,3-dichloro-1-propene, and then a decarboxylation takes place, converting the ester into the racemic 2-isopropyl-5-chloropent-4-enoic acid. A racemate separation is not described.

According to WO 2004/052828, the process from J. Agric. Food Chem. 32 (1), 1, pp. 85-92 is slightly modified in relation to some process parameters. This process again has the disadvantage of the racemate separation, described in the WO specification, by using the enzyme pig liver esterase (PLE).

An object of the present invention is to find a process for preparing enantiopure E-(2S)-alkyl-5-halopent-4-enoic acids and their esters which makes it possible to prepare the desired compounds in optical purities which are higher than in the prior art, of up to e.e. >99%, and in higher yields of up to 98% of theory, in a simple manner and avoiding pig liver esterase (PLE).

The present invention accordingly relates to a process for preparing enantiopure E-(2S)-alkyl-5-halopent-4-enoic acids and their esters of the formula (I)

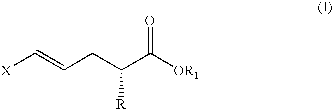

in which R is a C₁-C₆-alkyl radical, R₁ is H or C₁-C₄-alkyl and X is chlorine, bromine or iodine, which comprises a racemic 2-alkyl-5-halopent-4-enoic acid of the formula (II)

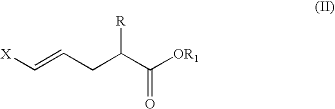

in which R and X are as defined above, and R₁ is H,
a) being reacted in a suitable solvent first with (S)-3-Methyl-2-phenylbutylamine, quinine or N-methyl-D-glucamine, and then
b) the corresponding 3-Methyl-2-phenylbutylamine salt, quinine salt or glucamine salt of the (R)-pentenoic acid being precipitated and removed, and
c) the remaining filtrate being mixed with a second chiral base or an inorganic salt, and then the desired salt of the (S)-pentenoic acid being precipitated, and
d) then converted into the corresponding E-(2S)-alkyl-5-halopent-4-enoic acid of the formula (I)

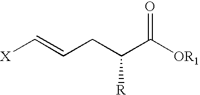

in which X and R are as defined above, and R₁ is H, and subsequently converted where appropriate into the corresponding ester of the formula (I) in which R₁ is C₁-C₄-alkyl.

Enantiopure E-(2S)-alkyl-5-halopent-4-enoic acids and their esters of the formula (I) are prepared by the process of the invention.

R in the formula (I) is a C₁-C₆-alkyl radical such as, for example, methyl, ethyl, n- and i-propyl, n-, i- and t-butyl, pentyl and hexyl.

C₁-C₄-alkyl radicals are preferred, and the i-propyl radical is particularly preferred.

R₁ in the case of the carboxylic acids is H and in the case of the esters is a C₁-C₄-alkyl radical, preferably a C₁-C₂-alkyl radical and particularly preferably a methyl radical.

X is chlorine, bromine or iodine, preferably chlorine.

The preparation according to the invention of the enantiopure (S)-carboxylic acids and their esters of the formula (I) takes place in a plurality of steps.

In the first step a) a racemic 2-alkyl-5-halopent-4-enoic acid of the formula (II) in which R and X are as defined above, and R₁ is H, reacted with (S)-3-Methyl-2-phenylbutylamine, quinine or N-methyl-D-glucamine.

Suitable starting compounds of the formula (II), can be prepared for example as in the prior art as described for example in J. Agric. Food Chem. 32 (1), 1, pp. 85-92, WO 2004/052828 or WO 01/09079.

Step a) is carried out in a suitable solvent.

Suitable solvents in this connection are ketones, esters (e.g. acetates), alcohols or ethers. Examples thereof are acetone, isopropyl acetate, methylisobutylcarbinol, tetrahydrofuran, etc.

Preferred solvents are acetates.

(S)-3-Methyl-2-phenylbutylamine, Quinine or N-methyl-D-glucamine are in this case added to the reaction solution composed of racemic acid of the formula (II) in the appropriate solvent. The amount of (S)-3-Methyl-2-phenylbutylamine, quinine or N-methyl-D-glucamine employed is from 0.5 to 1.2 mole equivalents, preferably 0.7 to 0.9 mole equivalents.

The addition takes place at a temperature of from 0 to 100° C., preferably from 60 to 80° C.

Subsequently, in step b) the reaction mixture is cooled to −10° C. to +10° C., preferably to −5° C. to +5° C. During this, the unwanted salt of (R)-pentenoic acid precipitates and is removed for example by filtration.

The filtrate remaining after removal of the (R)-salt, which now comprises almost exclusively the desired (S)-enantiomer of the carboxylic acid of the formula (I) is, where appropriate, first washed with acidic water having a pH below 7. The pH can in this case be adjusted with conventional acids such as, for example, HCl, H₂SO₄, etc.

Before further reaction with a second chiral base or the inorganic salt, where appropriate, part of the solvent is removed, for example by distillation.

In step c), a second chiral base or an inorganic salt is then added to the filtrate. Suitable as chiral base in this connection are conventional bases such as, for example (S)- or (R)-phenylethylamine, (S)-3-Methyl-2-phenylbutylamine, (L)- or (D)-pseudoephedrine, (L)- or (D)-norephedrine etc.

Examples of suitable inorganic salts are Li salts such as, for example, Li hydroxide, Li methoxide, etc.

The chiral base or the inorganic salt is used in this case in an amount of from 1 to 1.5 mole equivalents.

The reaction temperature in this step is from 0 to 100° C., preferably 60 to 80° C.

Subsequently in step c), the reaction mixture is cooled to −10° C. to +10° C., preferably to −5° C. to +5° C. During this, the corresponding salt of the (S)-pentenoic acid precipitates and is then isolated from the reaction mixture, for example, by filtration. To obtain the desired free (S)-acid of the formula (I), the salt is mixed with a water-immiscible solvent and extracted with acidic water. Examples of suitable solvents are esters (e.g. acetates), ethers (e.g. MTBE, THF, etc.), ketones (e.g. MIBK, etc.), alcohols (e.g. MIBC), hydrocarbons (e.g. hexane, toluene, etc.)

The corresponding enantiopure (S) acid of the formula (I) with R₁ equal to H is then obtained from the organic phase by concentration.

If the corresponding ester is the desired final product, the acid is converted into the desired ester.

This can take place for example in a C₁-C₄-alcohol, preferably in a C₁-C₂-alcohol and particularly preferably in methanol, in the presence of an acid such as, for example HCl, H₂SO₄, H₃PO₄, methanesulfonic acid, toluenesulfonic acid, trifluoroacetic acid etc., or of an acidic ion exchanger, the addition of the alcohol being followed first by distillation out of a mixture of alcohol and remaining solvent, and then by addition of a catalytic amount of one of the abovementioned acids.

The reaction temperature depends on the alcohol used and is from 50 to 100° C.

The temperature is preferably that of reflux, in which case alcohol is repeatedly added to the reaction mixture in approximately the amount distilled out as alcohol/water overhead.

After the reaction is complete, the reaction mixture is neutralized where appropriate with a base, for example with sodium methoxide, sodium hydroxide solution, KOH, K₂CO₃ etc., and the desired enantiopure E-(2S)-alkyl-5-halo-4-pentenoic esters are obtained with an e.e. of >99% and in a yield of >98% by distillation.

The esterification can, however, also take place by other conventional esterification methods, for example using SOCl₂/C₁-C₄-alcohol or using DMF-di-C₁-C₄-alkyl acetal.

The corresponding acids and esters of the formula (I) are obtained by the process of the invention in theoretical yields of up to 98% yield and with an e.e. of up to >99%, avoiding, inter alia, enzymes of animal origin.

### EXAMPLE 1

42.3 g (0.24 mol) of racemic 2-isopropyl-5-chloro-4-pentenoic acid were dissolved in 1337.5 ml of isopropyl acetate and heated to 60-70° C., and 67.8 g (0.20 mol) of quinine were added. The mixture was then cooled at a rate of 0.17° C./min until a turbidity resulted (58.5° C.) and was then cooled to 53.5° C. over the course of one hour. It was cooled further to 0° C. over the course of three hours and kept at 0° C. for one hour, during which the quinine salt of (R)-pentenoic acid precipitated. It was filtered off and washed once with cold (0° C.) isopropyl acetate (100 ml).

The remaining filtrate was washed first with 4% strength aqueous HCl (180 g) and then with water (90 g). Part of the isopropyl acetate (795 ml) was distilled at a max. 100° C. and then, at 60° C., 13.7 g (0.11 mol) of (S)-phenylethylamine were added. The resulting reaction mixture was cooled at a rate of 0.17° C./min until a turbidity resulted (56.2° C.) and was then cooled to 51.2° C. over the course of one hour. It was cooled further to 0° C. over the course of three hours and kept at 0° C. for one hour, during which the PE salt of the (S)-pentenoic acid precipitated. It was filtered off and washed with cold (−5° C.) isopropyl acetate (2×34 g).

36 g of PE salt washed with isopropyl acetate were suspended in 108 g of water, and 7.2 g of H₂SO₄ (76% strength) were added (pH of this solution 1.8). Then 65 g of isopropyl acetate were added, and the phases were separated. The organic phase was washed with 30 g of water, and the solvent was removed in vacuo. 16.6 g of the (S)-2-isopropyl-5-chloro-4-pentenoic acid were obtained as a colorless liquid in a yield of 85% of theory and with an optical purity of e.e.>98%.

1. A process for preparing enantiopure E-(2S)-alkyl-5-halopent-4-enoic acids and their esters of the formula (I) 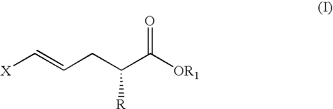
in which R is a.C₁C₆-alkyl radical, R₁ is H or C₁-C₄-alkyl and X is chlorine, bromine or iodine, which comprises a racemic 2-alkyl-5-halopent-4-enoic acid of the formula (II) 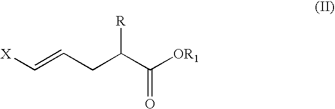
in which R and X are as defined above, and R₁ is H,
a) being reacted in a suitable solvent first with (S)-3-Methyl-2-phenylbutylamine, quinine or N-methyl-D-glucamine, and then
b) the corresponding (S)-3-Methyl-2-phenylbutylamine salt, quinine salt or glucamine salt of the (R)-pentenoic acid being precipitated and removed, and
c) the remaining filtrate being mixed with a second chiral base or an inorganic salt, and then the desired salt of the (S)-pentenoic acid being precipitated, and d) then converted into the corresponding E-(2S)-alkyl-5-halopent-4-enoic acid of the formula (I) 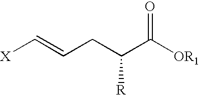
which X and R are as defined above, and R₁ is H, and subsequently converted where appropriate into the corresponding ester of the formula (I) in which R₁ is C₁-C₄-alkyl.

2. The process as claimed in claim 1, wherein a ketone, ester, alcohol or ether is used as solvent in step a).

3. The process as claimed in claim 1, wherein (S)-3-Methyl-2-phenylbutylamine, quinine or N-methyl-D-glucamine is added in an amount of from 0.5 to 1.2 mole equivalents in step a).

4. The process as claimed in claim 1, wherein step a) is carried out at from 0 to 100° C.

5. The process as claimed in claim 1, wherein the (S)-3-Methyl-2-phenylbutylamine salt, quinine or glucamine salt of the (R)-pentenoic acid is precipitated in step b) by cooling the reaction mixture to −10° C. to +10° C.

6. The process as claimed in claim 1, wherein the filtrate remaining after removal of the (R) salt is washed first with acidic water where appropriate before step c).

7. The process as claimed in claim 1, wherein (S)- or (R)-phenyl-ethylamine, (S)-3-Methyl-2-phenylbutylamine, (L)- or (D)-pseudoephedrine, (L)- or (D)-norephedrine is employed as second chiral base in step c).

8. The process as claimed in claim 1, wherein a lithium salt is employed as inorganic salt in step c).

9. The process as claimed in claim 1, wherein the addition of the second chiral base or of the inorganic salt in step c) takes place at from 0 to 100° C.

10. The process as claimed in claim 1, wherein the addition of the second chiral base or of the inorganic salt in step c) is followed by cooling the reaction mixture to −10° C. to +10° C., after which the corresponding salt of the (S)-pentenoic acid precipitates.

11. The process as claimed in claim 1, wherein to convert the salt of the (S)-pentenoic acid into the free (S)-pentenoic acid of the formula (I) with R₁ equal to H in step d), the salt is mixed with a water-immiscible solvent and extracted with acidic water, after which the desired free (S)-pentenoic acid of the formula (I) with R₁ equal to H is obtained by concentrating the organic phase.

12. The process as claimed in claim 1, wherein if the (S)-pentenoic ester of the formula (I) with R₁ equal to C₁C₄-alkyl is the desired final product, the (S)-pentenoic acid obtained in step d) is esterified in a C₁-C₄ alcohol in the presence of an acid or using a SOCl₂/C₁-C₄ alcohol or using DMF di-C₁-C₄-alkyl acetal.
